(19) United States
(12) Patent Application Publication
(10) Pub. No.: US 2023/0218169 A1
(43) Pub. Date: Jul. 13, 2023

(54) BRAIN IMAGING SYSTEM AND BRAIN IMAGING METHOD

(71) Applicant: A-moy Limited, Apia (WS)

(72) Inventor: FAN-PEI YANG, Hsinchu City (TW)

(21) Appl. No.: 18/123,410

(22) Filed: Mar. 20, 2023

Related U.S. Application Data
(63) Continuation-in-part of application No. 16/366,431, filed on Mar. 27, 2019.

(30) Foreign Application Priority Data
Mar. 30, 2018 (TW) 107111316

(51) Int. Cl. A61B 5/00; A61B 5/055; A61B 6/03; G06T 11/00; G06T 7/00; G06T 5/40

(52) U.S. Cl. CPC A61B 5/0042; A61B 5/0035; A61B 5/055; A61B 6/032; A61B 5/4088; G06T 11/005; G06T 7/0014; G06T 5/40; G06T 2207/20081

(57) ABSTRACT

A brain imaging system and a brain imaging method are provided. The brain imaging system includes a first imaging device, a second imaging device and a processor. The first imaging device captures a first brain image set by scanning a patient, and the second imaging device captures a second brain image set. The processor is configured to: pre-process and enhance first and second brain image sets; select first features that are optimal for estimating cerebral perfusion and select second features that are optimal for brain lesion identification; obtain, by performing calculations on first features, a plurality of brain perfusion indices; and identify, by inputting the second features to a third deep learning model having been trained, position information and volume information of one or more target brain lesions in the brain of the patient.

### CROSS-REFERENCE TO RELATED PATENT APPLICATION

This application is a continuation-in-part application of the U.S. application Ser. No. 16/366,431, filed on Mar. 27, 2019 and entitled “BRAIN IMAGING SYSTEM AND METHOD”, now pending, the entire disclosures of which are incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an imaging system and an imaging method, and more particularly to a brain imaging system and a brain imaging method.

### BACKGROUND OF THE DISCLOSURE

Nuclear magnetic resonance (NMR) is a non-invasive way to detect human bodies. It obtains variations of magnetic dipole moment of water molecules through transmitting and receiving radio frequency signals, and further to differentiate normal and tumor tissues by using contrast agents. The computerized tomography (CT) is used to obtain a two-dimensional image by having X-rays scanned through a human body. However, the two-dimensional image can only be interpreted by medical staff, which may adversely affect the precision and efficiency thereof.

### SUMMARY OF THE DISCLOSURE

In response to the above-referenced technical inadequacies, the present disclosure provides a brain imaging system and a brain imaging method.

In order to solve the above-mentioned problems, one of the technical aspects adopted by the present disclosure is to provide a brain imaging system, which includes a first imaging device, a second imaging device and a processor. The first imaging device is configured to capture a first brain image set by scanning a patient, and the first brain image set includes a plurality of first brain images that provides cerebral data representing a first contrast agent in a brain of the patient over time. The second imaging device is configured to capture a second brain image set by scanning the patient, and the second brain image set includes a plurality of second brain images that provides cerebral data representing a second contrast agent in the brain of the patient over time. The processor is electrically connected to the first imaging device and the second imaging device, and the processor is configured to: obtain, by performing an image pre-processing process on the first brain image set and the second brain image set, a first processed brain image set and a second processed brain image set; obtain, by performing an image enhancing process on the first processed brain image set and the second processed brain image set, a first enhanced brain image set and a second enhanced brain image set; select, by using a first deep learning model having been trained, first features from the first enhanced image set that are optimal for estimating cerebral perfusion; select, by using a second deep learning model having been trained, second features from the second enhanced image set that are optimal for brain lesion identification; obtain, by performing calculations on first features, a plurality of brain perfusion indices; and identify, by inputting the second features to a third deep learning model having been trained, position information and volume information of one or more target brain lesions in the brain of the patient.

In order to solve the above-mentioned problems, another one of the technical aspects adopted by the present disclosure is to provide a brain imaging method, including: configuring a first imaging device to capture a first brain image set by scanning a patient, in which the first brain image set includes a plurality of first brain images that provides cerebral data representing a first contrast agent in a brain of the patient over time; configuring a second imaging device to capture a second brain image set by scanning the patient, in which the second brain image set includes a plurality of second brain images that provides cerebral data representing a second contrast agent in the brain of the patient over time; and configuring a processor, which is electrically connected to the first imaging device and the second imaging device, to: obtain, by performing an image pre-processing process on the first brain image set and the second brain image set, a first processed brain image set and a second processed brain image set; obtain, by performing an image enhancing process on the first processed brain image set and the second processed brain image set, a first enhanced brain image set and a second enhanced brain image set; select, by using a first deep learning model having been trained, first features from the first enhanced image set that are optimal for estimating cerebral perfusion; select, by using a second deep learning model having been trained, second features from the second enhanced image set that are optimal for brain lesion identification; obtain, by performing calculations on the first features, a plurality of brain perfusion indices; and identify, by inputting the second features to a third deep learning model having been trained, position information and volume information of one or more target brain lesions in the brain of the patient.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 shows a block diagram of a brain imaging system according to one embodiment of the present disclosure;

FIG. 2 shows a flow chart of a brain imaging method according to one embodiment of the present disclosure;

FIG. 3 shows a detailed flowchart of step S12;

FIG. 4 shows a detailed flowchart of step S122;

FIG. 5 shows a detailed flowchart of step S13;

FIG. 6 is a schematic diagram showing a flow path of a contrast agent according to one embodiment of the present disclosure;

FIG. 7 is a curve diagram showing an accumulated concentration function of a contrast agent according to one embodiment of the present disclosure;

FIG. 8 is a curve diagram showing a residual concentration function of a contrast agent according to one embodiment of the present disclosure;

FIG. 9 shows a schematic diagram of a brain image according to one embodiment of the present disclosure; and

FIG. 10 shows another flow chart of a brain imaging method according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

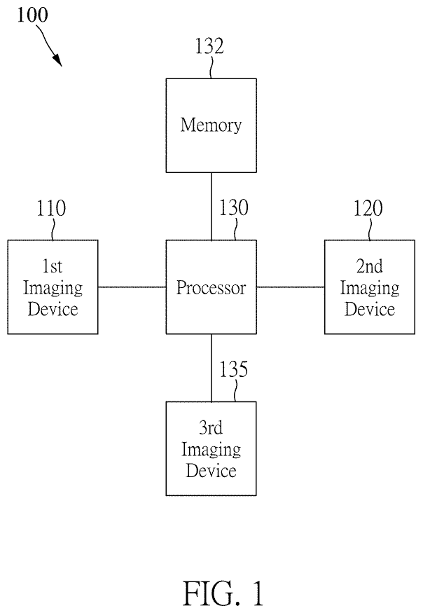

FIG. 1 shows a block diagram of a brain imaging system according to one embodiment of the present disclosure. Reference is made to FIG. 1, the present disclosure provides a brain imaging system 100 includes a first imaging device 110, a second imaging device 120, a third imaging device 135 and a processor 130, and the processor 130 is electrically connected to the first imaging device 110, the second imaging device 120 and the third imaging device 135.

The processor 130 can include one or more processing units, and can be, for example, a central processing unit (CPU) and/or a general-purpose microprocessor, a microcontroller, a digital signal processor (DSP), a field programmable gate array (FPGA), a programmable logic device (PLD), and a combination of any of the above devices that can perform data calculation or other operations, or any other suitable circuits, devices and/or structures.

The first imaging device 110 can be, for example, a computed tomography (CT) imaging device, which is configured to capture a first brain image set by scanning a patient. The first brain image set includes a plurality of first brain images that provides cerebral data representing a first contrast agent in a brain of the patient over time, and the first brain images can be, for example, CT brain images. In some embodiments, the first contrast agent can be, for example, an iodinated contrast agent. It should be noted that the brain images mentioned in the present disclosure generally refer to images captured with a field of view that covers the head and the neck of the patient.

Specifically, the second imaging device 120 can be, for example, a magnetic resonance imaging (MRI) device, which can be configured to capture a second brain image set by scanning the patient. The second brain image set includes a plurality of second brain images that provides cerebral data representing a second contrast agent in the brain of the patient over time, and the second brain images can be, for example, MRI brain images. In some embodiments, the second contrast agent can be, for example, a Gadolinium contrast agent. It should be noted that, the Gadolinium contrast agent can be, for example, Gadolinium-Diethylene Triamine Penta-acetic Acid (Gd-DTPA). Since Gd³⁺ in the lanthanide series is toxic and may lead to renal fibrosis as the excessive Gd³⁺ accumulates in human bodies, the Gd³⁺ is chelated by DTPA to form a stable compound, the Gd-DTPA.

Furthermore, the third imaging device 135 can be configured to capture a third brain image set. The third brain image set includes a plurality of third brain images, which can be, for example, structural brain images, such as T1 images. Specifically, an FMRIB Software Library (FSL) software can be executed by the third imaging device 135 or the processor 130, so as to capture the third brain images. Therefore, a cerebral cortex volume can be calculated to obtain a brain atrophy region. For example, the cerebral cortex may be the parietal cortex, the frontal lobe, the temporal cortex or the occipital lobe.

It should be noted that the processor 130 is electrically connected to a memory 132, and the memory 132 can be, for example, but not limited to, a hard disk, a solid-state disk, or other storage devices that can store data, which is configured to store at least a plurality of computer-readable instructions, the first brain image set, the second brain image set and the third brain image set mentioned above. In some embodiments, the processor 130 and the memory 132 can be included in a computing device, such as a general-purpose computer is one that, given the application and required time, should be able to perform the most common computing tasks. Desktops, notebooks, smartphones and tablets, are all examples of general-purpose computers.

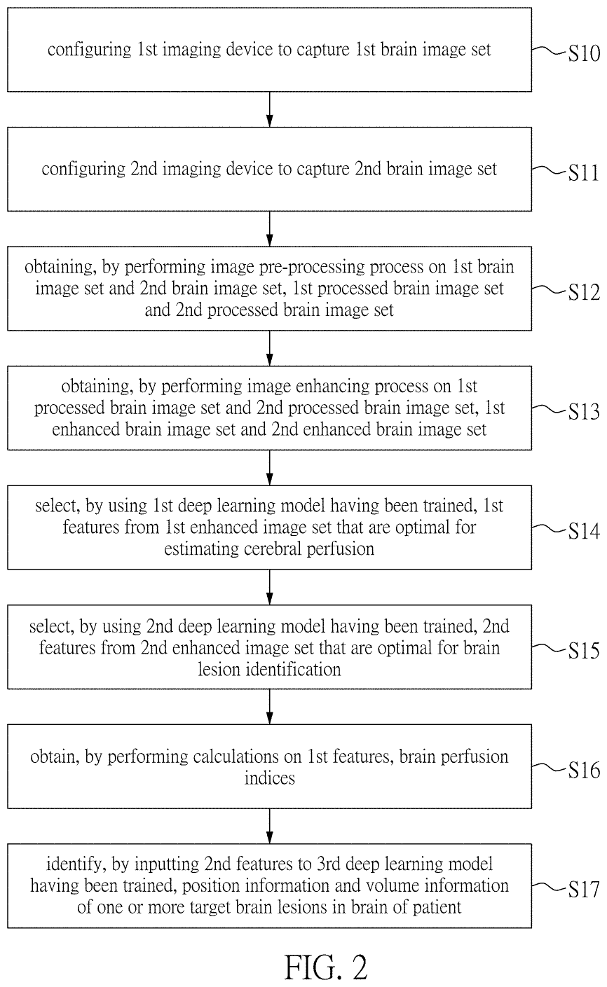

Reference is further made to FIG. 2, which shows a flowchart of a brain imaging method according to one embodiment of the present disclosure.

As shown in FIG. 2, the brain imaging method includes the following steps:

Step S10: configuring the first imaging device to capture the first brain image set. As mentioned above, the first brain image set can include CT brain images.

Step S11: configuring the second imaging device to capture the second brain image set. As mentioned above, the second brain image set can include MRI brain images.

The processor 130 is configured to, in response to receiving the first brain image set and the second brain image set, perform the following steps:

Step S12: obtaining, by performing an image pre-processing process on the first brain image set and the second brain image set, a first processed brain image set and a second processed brain image set.

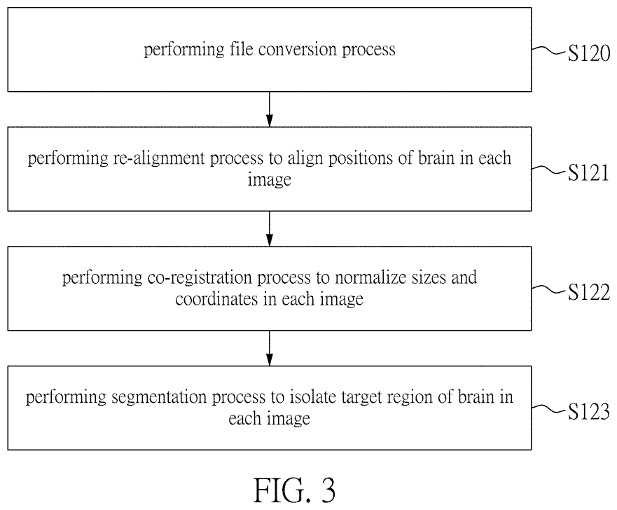

Reference is further made to FIG. 3, which shows a detailed flowchart of step S12. As shown, step S12 can include the following steps:

Step S120: performing a file conversion process. This step is performed for converting a file format of the first brain image set and the second brain image set into a format acceptable to subsequent image processing processes.

Step S121: performing a re-alignment process to align positions of the brain in each image.

Specifically, realignment in this step is performed to align all functional images in such a way that positioning of the brain in each image is the same. Although during data acquisition, head is packed with the padding or foam but still head movement is found, and such movement causes two major issues. Firstly, in a voxel the source of the signal can differ between scans over time which give fake activation. Secondly due to movement, signal to noise ratio (SNR) can be affected. Therefore, the re-alignment process can ensure that a source of signal in a specific voxel is always the same physical location, regardless of shaking conditions during brain image collection.

For the brain image set with severe head movement, multiple parameters, such as rotations (x, y and z-axis) and translations (left right, up down and forward backward), should be further considered in the subsequent image preprocessing, thereby increasing complexity of calculation.

Furthermore, realignment is an important step in the pre-processing of MRI data and it also has a key role in model estimation so it is better to consider realignment parameters as nuisance regressors. Although nuisance regressors are not part of statistical analysis and are considered as effect of no interest but they are very important in model estimation in reducing noise (error) and preparing the data for better statistical analysis.

Step S122: performing a co-registration process to normalize sizes and coordinates in each image.

Specifically, the co-registration process can provide the ability to geometrically align one dataset with another, and is a prerequisite for all imaging applications that compare datasets across subjects, imaging modalities, or across time. Registration algorithms also enable the pooling and comparison of experimental findings across laboratories, the construction of population-based brain atlases, and the creation of systems to detect group patterns in structural and functional imaging data.

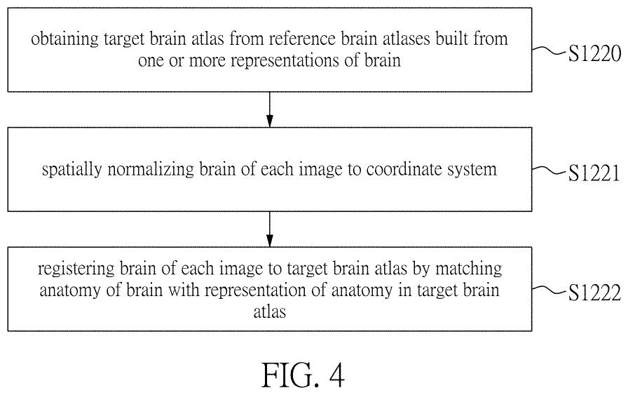

Reference can be made to FIG. 4, which shows a detailed flowchart of step S122. As shown, the co-registration process further includes:

Step S1220: obtaining a target brain atlas from a plurality of reference brain atlases built from one or more representations of brain.

In detail, brain atlases can be made from multiple modalities and individuals provide the capability to describe image data with statistical and visual power. The brain atlases have enabled a tremendous increase in the number of investigations focusing on the structural and functional organization of the brain. In humans and other species, the brain's complexity and variability across subjects is so great that reliance on atlases is essential to manipulate, analyze and interpret brain data effectively.

The reference brain atlases can include, for example, initially intended to catalog morphological descriptions, brain atlases based upon 3D tomographic images, anatomic specimens and a variety of histologic preparations that reveal regional cytoarchitecture, brain atlases that include regional molecular content such as myelination patterns, receptor binding sites, protein densities and mRNA distributions, and other brain atlases describe function, quantified by positron emission tomography, functional MRI or electrophysiology, and the target brain atlas can be selected from above examples of the reference brain atlases.

Step S1221: spatially normalizing the brain of each image to a coordinate system.

In this step, the coordinate system can be created to equate brain topology with an index must include carefully selected features common to all brains. Further, these features must be readily identifiable and sufficiently distributed anatomically to avoid bias. Once defined, rigorous systems for matching, or spatially normalizing a brain to this coordinate system can be further developed, thereby allowing individual data to be transformed to match the space occupied by the target brain atlas.

Step S1222: registering the brain of each image to the target brain atlas by matching anatomy of the brain with a representation of anatomy in the target brain atlas.

In this step, registration is performed to compare one brain atlas with another. The success of any brain atlas depends on how well the anatomies of individual subjects match the representation of anatomy in the atlas. While registration can bring the individual into correspondence with the atlas, and a common coordinate system enables the pooling of activation data and multi-subject comparisons.

Referring back to FIG. 3, the image pre-processing process proceeds to step S123: performing a segmentation process to isolate a target region of the brain in each image.

In this step, a binarization method can be utilized, in which gray matter and white matter of the brain can be manually or automatically selected with a mask, and structures such as braincase and ventricles can be excluded For structures to be excluded by masking, pixel color values are 0 after the binarization, and for the target region to be retained, pixel color values are 1 multiply by original pixel values.

Specifically, the segmentation is an important stage of the image recognition system, because it extracts the objects of interest, for further processing such as description or recognition. Segmentation techniques are used to isolate the target region from the brain images in order to perform analysis.

Referring back to FIG. 2, the brain imaging method proceeds to step S13: obtaining, by performing an image enhancing process on the first processed brain image set and the second processed brain image set, a first enhanced brain image set and a second enhanced brain image set.

In the case of high contrast or poor contrast in MRI images, there will be differences in determining brain lesions. Therefore, it needs to enhance the brain images to distinguish tumor cells from healthy cells, or to make the stroke infarction brighter, for example.

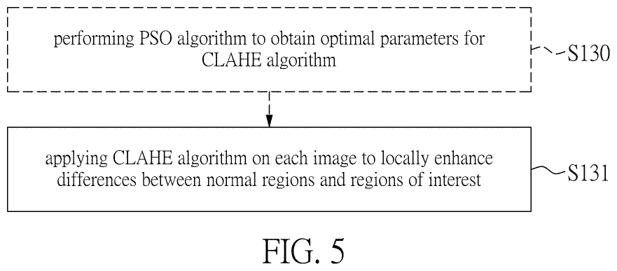

Reference is made to FIG. 5, which shows a detailed flowchart of step S13. As shown, the image enhancing process can further include the following steps:

Step S131: applying a contrast-limited adaptative histogram equalization (CLAHE) algorithm on each image to locally enhance differences between normal regions and regions of interest.

Normally, a histogram equalization (HE) algorithm can be used to enhance images by effectively spreading out the most frequent intensity values, that is, stretching out an intensity range of an image. This algorithm usually increases a global contrast of an image when its usable data is represented by close contrast values. This allows for areas of lower local contrast to gain a higher contrast.

In the case that the HE algorithm is performed for MRI images, it is found that bright parts of a brain image applied by the HE algorithm is overexposed, original details thereof are lost, and contrast of background noise is increased and useful signals are reduced since there are many dark parts in the MRI images.

Therefore, in step S131, the CLAHE algorithm is utilized to achieve local equalization on each image of the first brain image set, the second brain image set and the third brain image set that are processed by the pre-processing process. Different from the HE algorithm, the CLAHE algorithm provide local equalization for the brain image, such that bright parts can be well preserved and will not be overexposed due to equalization, and the noise of the latter will be less than that of the former. It should be noted that, tumor cells are easier to be distinguished from healthy cells in the brain images equalized by the CLAHE algorithm.

In the CLAHE algorithm, local enhancement of the MRI image is performed by dividing image into equal sized, distinct contextual regions or blocks and local histogram of each block is computed. The cumulative probability density for each block is computed and the clip limit for each block is computed based on Eq.(1). The user defined constant is in range of 0-1. On basis of clip limit (CL), the local histogram of each block is clipped. This clip limit is proportional to product of average height of the histogram for every block and a user defined constant α, in the range of 0-1. The average height of the histogram is ratio of size of the block to the number of gray levels. The clipping level, represented as CL for a block size of m*n pixels for L levels of gray level is given in the following equation:

CL=αMN/L.

The original height hₖ of the local histogram is replace with CL, as can be seen in the following equation:

$${h}_{k} = \{ \begin{matrix}CL , if {h}_{k} > CL \\ {h}_{k} , otherwise\end{matrix} ;$$

hₖ is the histogram of the block, and

Σₖ₌₀^{L-1}hₖ=MN.

The number of clipped pixels represented as n_{c} is computed using the following equation:

n_{c}=MN−Σₖ₌₀^{L-1}hₖ.

The clipped portion of the histogram is uniformly distributed to all the histogram bins to obtain the enhanced histogram. The enhanced histogram is renormalized to get its area under the curve. The distribution of the clipped portion can be uniform or non-uniform, and the distribution of the clipped pixels should not exceed the clipping level. The number of pixels distributed in each histogram bin is calculated using the following equation:

nₙ=n_{c}/L.

The enhanced histogram hₑ is given as the following equation:

$${h}_{e} = \{ \begin{matrix}CL , if {h}_{k} + {n}_{c} \geq CL \\ {h}_{k} + {n}_{c} , otherwise\end{matrix} .$$

This process is repeated until all clipped pixels are distributed uniformly. Subsequently, the cumulative histogram of the block, followed by histogram matching is performed, since the shape of the histogram greatly reflects the brightness and visual characteristics of the image. Histogram matching allows enhancing or degrading the brightness on the enhanced histogram to match the user-specified probability distribution. Therefore, the first enhanced brain image set and the second enhanced brain image set can be obtained.

In more detail, a level of clipping that can be adjusted in the CLAHE algorithm depends on a predefined parameter called clip limit. The clip limit is defined as a multiple of an average height of a histogram, calculated prior to computing a cumulative distribution function. Another significant parameter of the CLAHE algorithm is a block size that represents the number of pixels considered in each block. For example, for a block size of 8×8, 64 pixels are considered. The block size also affects the contrast of images significantly. The local contrast is achieved with smaller block size and the global contrast in the image is attained by larger block size. Hence, there is a trade-off in selection of block size.

Moreover, the distribution function for the CLAHE algorithm ranges from Uniform, Exponential and Rayleigh distribution. The choice of function is significant in determining the contrast of the image and depends on the performance parameters like entropy, standard deviation, peak signal to noise ratio and structural information of the image.

However, brain images may be different in various aspects, such as environments, thus the operational parameters, including clip-limit, block-size and distribution function should be selected empirically.

The clipping level represented as the average height of all the local histograms, in the above equation is not an accurate measure for all histograms or all images. Hence, there is a need to find a clipping level that is unique to every local histogram, thereby providing precise contrast enhancement.

Therefore, before applying the CLAHE algorithm, the image enhancing process can proceed to step S130 in advance: performing a particle swarm optimization (PSO) algorithm to obtain optimal parameters for the CLAHE algorithm.

Depending on the average height of the local histogram, the range for the values of clip limit can be specified by the user. The PSO algorithm helps in automatic selection of clip limit from a group of probable values. This group of probable values for clip limit is called the swarm and the individual elements of the swarm is called a particle. Every particle in the swarm is represented in terms of two parameters called position and velocity; to begin with, the position is initialized as 0 and velocity is initialized with some random values. The velocity and position of each particle is updated based on the fitness function. The quality of enhanced image is measured as a multi-objective function also called as a fitness function given in the following equation:

F(Iₑ)=log(log(E(Iₛ)))*n_edges(Iₛ)/(M*N)*H(Iₛ);

The fitness function F(Iₑ) is a product of entropy, sum of edge intensities and number of edge pixels, hence called multi-objective function. E(Iₛ) represents a sum of edges derived from Sobel edge operator, and H(Iₛ) represents the entropy value. The velocity and position of the particle can be updated. In every iteration, a fitness value is computed based on the particle's velocity and position. The particle that generates a maximum fitness value is represented as ‘pbest’. The procedure is repeated for a specified number of iterations, among all iterations, maximum of the ‘pbest’ value is represented as a ‘gbest’. The process of finding the optimal clipping level continues until the maximum value for the fitness function is achieved or until the iterations are exhausted. Therefore, the contrast of the brain image can be enhanced based on the information content and edge information computed from the fitness function. It can be seen that the enhanced brain images will have more of number of edge pixels, increased entropy as well as enhanced contrast.

Reference to FIG. 2 again, the brain imaging method proceeds to step S14.

Step S14: select, by using a first deep learning model having been trained, first features from the first enhanced image set that are optimal for estimating cerebral perfusion.

Step S15: select, by using a second deep learning model having been trained, second features from the second enhanced image set that are optimal for brain lesion identification.

In steps S14 and S15, the first deep learning model and the second deep learning model can be a first long short-term memory (LSTM) neural network and a second LSTM neural network, respectively, which are capable of learning order dependence in fitting time-series data.

For example, in the present embodiment, since perfusion data provided by the first brain set is sequential or temporal, the first LSTM neural network can be a first recurrent neural network (RNN) with an LSTM architecture, which is trained to filter usable features for estimating brain perfusion indices. For example, the first brain images can be CT perfusion sequential images, which are included in each sample input vector jointly with patient-specific information and a value or values for one or more injection protocol parameters. The ground truth provided for each sample in training data include a perfusion parametric image, color-map image, quantitative values such as peak value, time to peak, cerebral blood flow (CBF), and/or cerebral blood volume (CBV), and/or cardiologist or radiologist decision (e.g., diagnosis and/or therapy), but the present disclosure is not limited thereto.

Specifically, the LSTM neural network is a type of RNN capable of learning order dependence in sequence prediction problems. As a consequence, it is also largely used to fit time-series data. An LSTM has a chain structure that includes four neural networks and several memory units known as cells. First, significant information is added to the neuron via the input gate. The forget gate then removes information that is no longer helpful in the present neuron state. The bias is applied to the current and prior inputs after they have been multiplied by the weight matrices. The result is fed into a binary activation function (similar to sigmoid). If the output state is zero, the information is deleted. If the output state is one, the information is saved for later use. Finally, the output gate is in charge of retrieving useful data from the neuron and sending it to the next neuron.

Therefore, in this case, the most significant features in the CT perfusion sequential images (i.e., the first brain image set) can be filtered by the first LSTM neural network without sacrificing the accuracy of the real data pattern and make it useful to forecast a time series. On the basis of the time-series tables and figure plots, the LSTM-related models fit better to the data patterns, while, on the other hand, the probabilistic models were able to better capture the spike points. That is, the probabilistic approaches during the learning phase focus mostly on the peak locations, whereas the LSTM-related models focus on the growth and decay elements of the curves.

Similarly, the second LSTM neural network can be a second recurrent neural network (RNN) with an LSTM architecture, which is trained to filter usable features for identifying possible brain lesions, such as infarction areas, tumors, tumor metastasis, lymph nodes and lesions associated with dementia.

Therefore, the most significant features in the MRI perfusion sequential images (i.e., the second brain image set) can be filtered by the second LSTM neural network.

Step S16: obtain, by performing calculations on the first features, a plurality of brain perfusion indices.

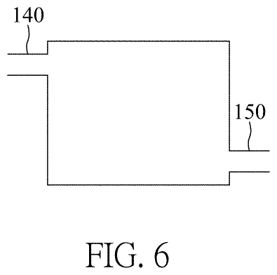

For better understanding, the first features are simplified to a first concentration curve by Hounsfield Unit (HU). Reference can be made to FIG. 6, which is a schematic diagram showing a flow path of contrast agents according to one embodiment of the present disclosure. As shown in FIG. 6, a simple model representing the first contrast agent flowing in the brain of the patient is provided. In FIG. 6, positions of an entrance 140 and an exit 150 where the contrast agent flows into and out from the brain can identified from the first brain image set, such that the first concentration curve (i.e., a concentration curve of an iodinated contrast agent) can be obtained. In this embodiment, the first concentration curve is a concentration curve of an iodinated contrast agent, and the first contrast agent time to peak is an iodinated contrast agent time to peak. A slope of the concentration curve of the iodinated contrast agent is positively proportional to Hounsfield Unit. The processor 130 detects the position of the entrance 140 of the brain according to an iodinated contrast agents starting time, an iodinated contrast agents time to half-peak and the iodinated contrast agent time to peak.

Therefore, the brain perfusion indices, including one or more of a cerebral blood flow (CBF), a cerebral blood volume (CBV), a cerebral blood mean transit time (MTT) and a first contrast agent time to peak (TTP) can be calculated and obtained according to the first concentration curve, but the present disclosure is not limited thereto.

Afterward, a vessel occlusion, infarction or ischemia region of the first brain image set can be detected according to one of the cerebral blood flow, the cerebral blood volume, the cerebral blood mean transit time and the first contrast agent time to peak. Specifically, when the cerebral blood flow is below 30% of a normal cerebral blood flow, the cerebral blood volume is smaller than 40% of a normal cerebral blood volume and the first contrast agent time to peak is increasing, the processor 130, through the first imaging device 110, detects an infarct core of the vessel occlusion, infarction or ischemia region in the first brain image. In addition, when the cerebral blood flow is decreasing, the cerebral blood volume is maintained or increased, and the first contrast agent time to peak is dramatically increasing, the processor 130 through the first imaging device 110 detects a penumbra of the vessel occlusion, infarction or ischemia region in the first brain image.

Step S17: identify, by inputting the second features to a third deep learning model having been trained, position information and volume information of one or more target brain lesions in the brain of the patient.

Similarly, in this embodiment, the second features are simplified into a second concentration curve, which is a concentration curve of a Gadolinium contrast agent, and the second contrast agent time to peak is a Gadolinium contrast agent time to peak. As can be seen from FIG. 6, the position of the entrance 140 of the brain can be similarly obtained according to a Gadolinium contrast agent starting time, a Gadolinium contrast agent time to half-peak and the Gadolinium contrast agent time to peak. It should be noted that, the Gadolinium contrast agent can be Gadolinium-DiethyleneTriamine Penta-acetic Acid (Gd-DTPA). Since Gd³⁺ in the lanthanide series is toxic and may lead to renal fibrosis as the excessive Gd³⁺ accumulates in human bodies, the Gd³⁺ is chelated by DTPA to form a stable compound, the Gd-DTPA.

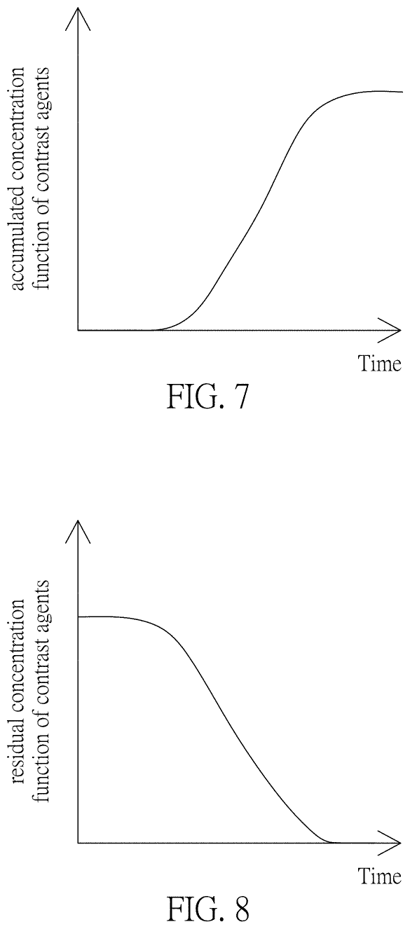

Reference can be made to FIGS. 7 and 8, FIG. 7 is a curve diagram showing an accumulated concentration function of a contrast agent according to one embodiment of the present disclosure, and FIG. 8 is a curve diagram showing a residual concentration function of a contrast agent according to one embodiment of the present disclosure. FIGS. 7 and 8 show that, the accumulated concentration function of the contrast agent increases but the residual concentration function of the contrast agent decreases with time.

Furthermore, the CBF, the CBV, the MTT and the second contrast agent TTP can be calculated and obtained according to the second concentration curve, and can be included in the second features. Therefore, in step S17, position information and volume information of the target brain lesion, such as a vessel occlusion, infarction or ischemia region of the second brain image set, can be obtained through the third deep learning model having been trained according to one of the second concentration curve, the cerebral blood flow, the cerebral blood volume, the cerebral blood mean transit time and the second contrast agent time to peak. However, the aforementioned description for the second features is merely an example, and is not meant to limit the scope of the present disclosure.

In more detail, a plurality of candidate deep learning models can be trained for identifying different types of brain lesions. The candidate deep learning models can include, for example, models such as YOLO and faster R-CNN. When identifying and calculating a volume of brain lesions, object detection and object recognition needs to be performed at once or in sequence, that is, one-stage or two-stage manner.

In order to get better identification results and accuracy, each type of brain lesions is trained separately since the brain lesions may be too similar to one another. Therefore, the candidate deep learning models are trained by training sets having different types of images, respectively.

The different types of images can include, for example, diffusion weighted images (DWI), apparent diffusion coefficient (ADC) images, and T2-FLAIR images.

Furthermore, the diffusion weighted images can be obtained according to the following equation:

$$S ( x , y , b ) = {M}_{0} ( \begin{matrix}1 - & {e}^{- \frac{TR}{{T}_{1} ( x , y )}}\end{matrix} ) {e}^{- \frac{T E}{{T}_{2}^{*} ( x , y )}} {e}^{- ADC \cdot b} ;$$

T₁ is a spin-lattice relaxation time, T₂* is a transverse relaxation time, TR is a cycle time, TE is an echo time, b is a setting parameter of an imaging device, (x, y) is a position of the brain image, and M₀ is an initial value of the brain image when time is zero). In practice, “b” can be set as 0 or 1000. T₁ is parallel with a magnetic field orientation. When a magnetic dipole moment is opposite to the magnetic field orientation, the magnetic dipole moment has a maximum energy. On the other hand, when the magnetic dipole moment and the magnetic field orientation are the same, the magnetic dipole moment has a minimum energy. T₂* is vertical to the magnetic field orientation. Generally, a substance includes magnetic dipole moments, and each of them has different energy with respect to the magnetic field. Some magnetic dipole moments have higher energy, and some magnetic dipole moments have lower energy. The vector sum of all the magnetic dipole moments will gradually decrease, and a decreasing rate can be represented by the transverse relaxation time T₂*.

Therefore, the DWIs can be obtained. A substance diffusion is 3-dimensional. A diffusion of water molecules may be affected by the surroundings and other molecules close to them, and thus the diffusion of water molecules is anisotropic. Fractional anisotropy (FA) is a value to evaluate the anisotropy of the molecule diffusion. The FA is a value from 0 to 1, wherein “1” indicates a high degree of anisotropy but “0” indicates a low degree of anisotropy. For example, a white matter has a high degree of anisotropy, but a grey matter has a low degree of anisotropy.

Furthermore, an apparent diffusion coefficient (ADC) can be calculated as follows to obtain the ADC images:

$$A D C ( x , y ) = - \frac{1}{b} ln ( \frac{S ( x , y , 1000 )}{S ( x , y , 0 )} ) {mm}^{2} / s .$$

In some embodiments, for each type of the brain lesions, the candidate deep learning models, such as YOLO and faster R-CNN models, are trained by the DWI and ADC images, and the trained candidate deep learning models are each tested to determine whether or not each of the candidate deep learning models can be selected to identify the one or more target brain lesions. However, the aforementioned description for the candidate deep learning models is merely an example, and is not meant to limit the scope of the present disclosure.

The YOLO (You Only Look Once) model used herein is a single-stage object detection algorithm that predicts the bounding boxes and class probabilities of objects in a single forward pass through the neural network. The YOLO algorithm at least includes a step of dividing an input brain image into a grid of cells, in which each cell is responsible for predicting a fixed number of bounding boxes and their associated class probabilities and each bounding box prediction consists of a set of values including x, y, width, height, and confidence score. Furthermore, non-maximum suppression is used to eliminate redundant bounding box predictions. The YOLO model consists of a convolutional neural network (CNN) that extracts features from the input image, followed by several fully connected layers that make the final predictions.

The Faster R-CNN models is a two-stage object detection algorithm that first generates region proposals before predicting class probabilities and refining bounding boxes. The Faster R-CNN algorithm at least includes steps of passing an input brain image is through a CNN to extract feature maps, generating candidate regions that may contain objects by using a region proposal network (RPN), pooling and feeding the candidate regions into a classifier to predict the class probabilities and refine the bounding boxes, and eliminating redundant bounding box predictions by using non-maximum suppression.

The Faster R-CNN model has two parts, a CNN that extracts features from the input image and an RPN that generates candidate regions for further processing. The RPN is trained to distinguish between foreground and background regions and to generate high-quality region proposals. The classifier is trained to classify the regions and refine the bounding boxes.

Assuming that there are 30 to-be-tested brain images, which are input to candidate deep learning models trained with DWI and ADC images to generate detection results, such as DWI-1 and ADC-1, DWI-2 and ADC-2 . . . , DWI-30 and ADC-30, in which the target brain lesion is detected. The corresponding two detection results, such as DWI-1 and ADC-1, are compared to determine whether or not the detected target brain lesions in the two detection results having the same volumes and at the same positions, thereby determining whether or not the candidate deep learning model can be used to identify the one or more target brain lesions.

Therefore, according to the type of the target brain lesions, the third deep learning model can be selected from the candidate deep learning models having been trained for identifying different types of brain lesions.

Furthermore, parameters of the candidate deep learning models having been trained can also be used to determine whether or not the candidate deep learning model can be used to identify the one or more target brain lesions. The parameters can include, for example, precision, recall, mean average precision (mAP) and other metric used to evaluate object detection models. In some embodiments, the one or more target brain lesions can include one or more of infarction areas, tumors, tumor metastasis, lymph nodes and lesions associated with dementia.

In addition to using the machine learned model, in the present disclosure, the infarct core of the vessel occlusion, infarction or ischemia region in the second brain image set can be detected by the processor 130 when the ADC is smaller than a diffusion threshold, or a penumbra of the vessel occlusion, infarction or ischemia region in the second brain image set can be detected by the processor 130 when the second contrast agent time to peak is larger than a time to peak. In practice, the ADC should be divided by 1,000,000, and the position of the brain image (x, y) includes two algebras referring to the position of the brain image. For example, the diffusion threshold can be 600 mm²/s, and the time to peak can be 6 seconds. The diffusion threshold and the time to peak can be further calculated by the processor 130 based on Bayesian statistics, but values are not limited in the present disclosure.

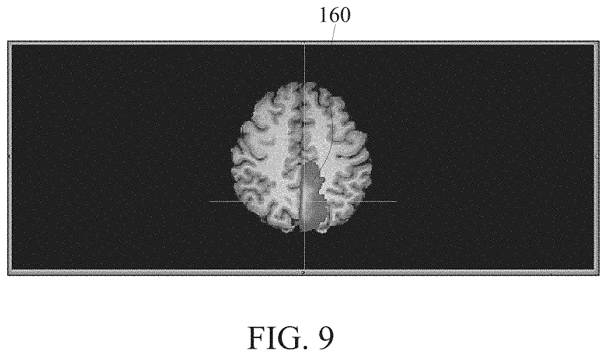

Reference is made to FIG. 9, which shows a schematic diagram of a brain image according to one embodiment of the present disclosure. In FIG. 9, the processor 130 can execute an FMRIB Software Library (FSL) software to perform a Brain Extraction Tool to capture a calvarium image of the second brain image and separate the calvarium image from the second brain image. Then, the processor 130 divides the second brain image without the calvarium image into a plurality of brain regions. The processor 130 detects a penumbra 160 of the vessel occlusion, infarction or ischemia region in the second brain image based on the Bayesian statistics. Specifically, according to a FSL instruction, the processor 130 divides the second brain image without the calvarium image into 15 brain regions, wherein these 15 brain regions include left brain regions and right brain regions. When the processor 130 receives the FSL instruction, the processor 130 uses the FSL software to do calculations for the cortex division, positions of the brain regions and volumes of the brain regions. The diffusion thresholds of the brain regions are different. Also, the diffusion thresholds of the brain regions may be varied due to age, gender or brain diseases. The processor 130 can determines the diffusion thresholds of all brain regions based on a big data analysis (e.g., the Bayesian statistics) to detect the penumbra 160 of the vessel occlusion, infarction or ischemia region in the second brain image. Therefore, by using the FSL software, the processor 130 can not only divide the calvarium image from the second brain image, but also can calculate the volume of each brain region.

The processor 130 uses algorithms to generate an image of the vessel occlusion, infarction or ischemia region according to the vessel occlusion, infarction or ischemia region in the first brain image and the vessel occlusion, infarction or ischemia region in the second brain image. Specifically, the first brain image is the CT brain image, and the second brain image is the MRI brain image. Although the CT brain image has a low resolution with respect to the substantia nigra and the substantia alba, the CT brain image costs less time to be measured, being able to rapidly detect the vessel occlusion, infarction or ischemia region. On the other hand, the MRI brain image costs more time to be measured although the MRI brain image has a high resolution with respect to the substantia nigra and the substantia alba, which helps to find the long term vessel occlusion, infarction or ischemia region and pathological changes around the vessel occlusion, infarction or ischemia region. In short, the CT brain image and the MRI brain image help detect the vessel occlusion, infarction or ischemia region, but both have pros and cons. Therefore, the present disclosure uses the algorithms to generate the image of the vessel occlusion, infarction or ischemia region according to the vessel occlusion, infarction or ischemia region detected by the CT and the MRI. Additionally, the present disclosure uses a set-up application to automatically examine whether there is a vessel occlusion, infarction or ischemia in a patient's brain, so that the medical staff would not need to determine whether there is a vessel occlusion, infarction or ischemia in a patient's brain by observing the brain image.

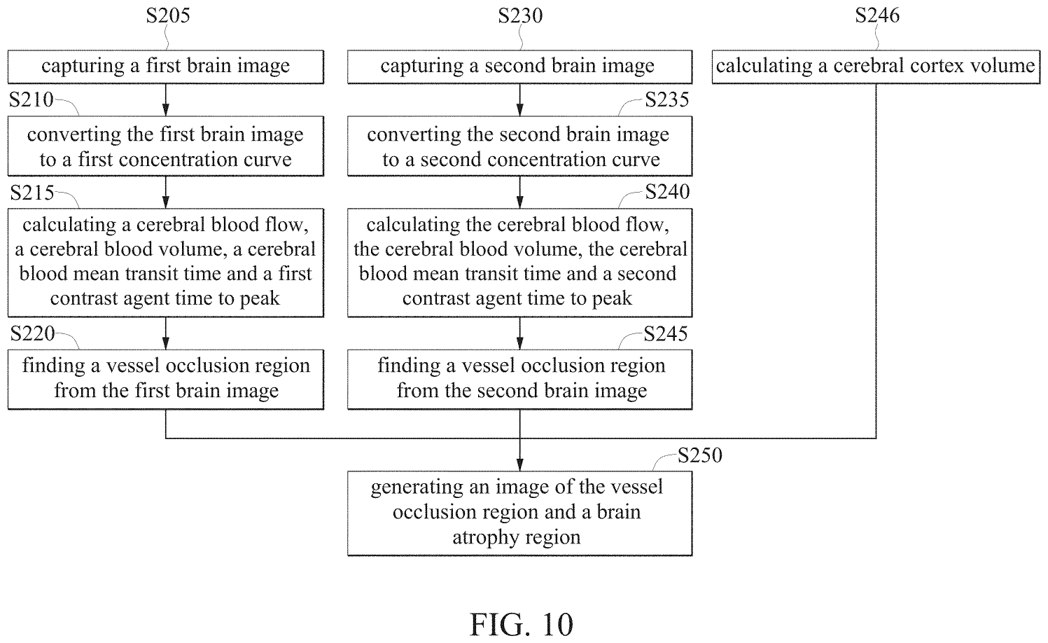

FIG. 10 shows another flowchart of a brain imaging method according to one embodiment of the present disclosure. The brain imaging method is adapted to the brain imaging system 100 to capture the brain image by using the contrast agent. According to FIGS. 1 and 6, the brain imaging system 100 can detect the entrance 140 and the exit 150 of the brain where the contrast agent flows into and out from the brain.

In step S205, the first imaging device 110 captures the first brain image set.

In step S210, the processor 130 is configured to convert the first brain image set to the first concentration curve by the Hounsfield unit. The first concentration curve is the concentration curve of the iodinated contrast agent, and the first contrast agent time to peak is the iodinated contrast agent time to peak. In addition, the slope of the concentration curve of the iodinated contrast agents is positively proportional to the Hounsfield unit.

In step S215, the processor 130 is configured to calculate the cerebral blood flow, the cerebral blood volume, the cerebral blood mean transit time and the first contrast agent time to peak according to the first concentration curve showing the concentrations at the positions of the entrance 140 and the exit 150. Also, the processor 130 is further configured to detect the position of the entrance 140 iodinated according to the iodinated contrast agent starting time, the iodinated contrast agent time to half-peak and the iodinated contrast agent time to peak.

In step S220, the processor 130 detects the vessel occlusion, infarction or ischemia region of the first brain image according to one of the cerebral blood flow, the cerebral blood volume, the cerebral blood mean transit time and the first contrast agent time to peak. Specifically, when the cerebral blood flow is below 30% of the normal cerebral blood flow, the cerebral blood volume is smaller than 40% of the normal cerebral blood volume and the first contrast agent time to peak is increasing, the processor 130 through the first imaging device 110 detects the infarct core of the vessel occlusion, infarction or ischemia region in the first brain image. In addition, when the cerebral blood flow is decreasing, the cerebral blood volume is maintained or increased, and the first contrast agent time to peak is dramatically increasing, the processor 130 through the first imaging device 110 detects the penumbra of the vessel occlusion, infarction or ischemia region in the first brain image.

In step S230, the second imaging device 120 captures the second brain image set.

In step S235, the processor 130 is configured to convert the second brain image to the second concentration curve through an equation (shown in the above embodiment). The second concentration curve is the concentration curve of the Gadolinium contrast agent, and the second contrast agent time to peak is the Gadolinium contrast agent time to peak.

In step S240, the processor 130 is configured to calculate the cerebral blood flow, the cerebral blood volume, the cerebral blood mean transit time and the second contrast agent time to peak according to the second concentration curve showing the concentrations at the positions of the entrance 140 and the exit 150. Also, the processor 130 the processor 130 is configured to detect the position of the entrance 140 where the Gadolinium contrast agent flows into the brain according to the Gadolinium contrast agent starting time, an Gadolinium contrast agents time to half-peak and the Gadolinium contrast agent time to peak.

In step S245, the processor 130 detects the vessel occlusion, infarction or ischemia region of the second brain image according to one of the second concentration curve, the cerebral blood flow, the cerebral blood volume, the cerebral blood mean transit time and the second contrast agent time to peak.

In step S246, the third imaging device 135 uses the FSL software to capture the third brain image. The third brain image is a structural brain image, and the structural brain image is the T1 image. In addition, the third imaging device 135 can be also configured to calculate the cerebral cortex volume to obtain the brain atrophy region.

In step S250, the processor 130 uses the algorithms to generate the images of regions with the vessel occlusion, infarction or ischemia according to the vessel occlusion, infarction or ischemia region in the first brain image and the vessel occlusion, infarction or ischemia region in the second brain image. In addition, the third imaging device 150 calculates the brain atrophy region according to the cerebral cortex volume.

In conclusion, in the present disclosure, the CT brain image set and the MRI brain image set are captured respectively by the first imaging device and the second imaging device. Then, the CT brain image set and the MRI brain image set are pre-processed and enhanced, in which the CLAHE algorithm provide local equalization for the brain image by using the optimal parameters that are obtained by the PSO algorithm. Moreover, the LSTM model is further utilized to filter features from the enhanced brain image sets that are optimal for estimating cerebral perfusion and brain lesion identification, without sacrificing the accuracy of the real data pattern and make it useful to forecast a time series.

In another aspect of the brain imaging system and the brain imaging method provided by the present disclosure, the CT and MRI brain image sets are converted into the concentration curves to calculate the cerebral blood flow, the cerebral blood volume, the cerebral blood mean transit time and the contrast agent time to peak. After that, the processor detects the vessel occlusion, infarction or ischemia region in the CT brain image, and the vessel occlusion, infarction or ischemia region and regions where blood flows are affected in the MRI brain image according to the cerebral blood flow, the cerebral blood volume, the cerebral blood mean transit time and the contrast agent time to peak. In addition, the third imaging device calculates the cerebral cortex volume to determine whether a specific brain region has obvious atrophy or affections. The images of regions with the vessel occlusion, infarction or ischemia and the brain atrophy region are generated by algorithms to improve the conventional way of determining the positions of the vessel occlusion, infarction or ischemia region and the brain atrophy region. Therefore, the present disclosure effectively improves the efficiency and the precision of the examination of the brain vessel occlusion and dementia.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A brain imaging system, comprising:
a first imaging device, configured to capture a first brain image set by scanning a patient, wherein the first brain image set includes a plurality of first brain images that provides cerebral data representing a first contrast agent in a brain of the patient over time;
a second imaging device, configured to capture a second brain image set by scanning the patient, wherein the second brain image set includes a plurality of second brain images that provides cerebral data representing a second contrast agent in the brain of the patient over time; and
a processor electrically connected to the first imaging device and the second imaging device, wherein the processor is configured to:
obtain, by performing an image pre-processing process on the first brain image set and the second brain image set, a first processed brain image set and a second processed brain image set;
obtain, by performing an image enhancing process on the first processed brain image set and the second processed brain image set, a first enhanced brain image set and a second enhanced brain image set;
select, by using a first deep learning model having been trained, first features from the first enhanced image set that are optimal for estimating cerebral perfusion;
select, by using a second deep learning model having been trained, second features from the second enhanced image set that are optimal for brain lesion identification;
obtain, by performing calculations on first features, a plurality of brain perfusion indices; and
identify, by inputting the second features to a third deep learning model having been trained, position information and volume information of one or more target brain lesions in the brain of the patient.

2. The brain imaging system according to claim 1, wherein the first imaging device is a computed tomography (CT) imaging device, the plurality of first brain images are CT brain images, the second imaging device is a magnetic resonance imaging (MRI) device, and the plurality of second brain images are MRI brain images.

3. The brain imaging system according to claim 2, wherein the pre-processing process includes:
performing a re-alignment process to align positions of the brain in each image;
performing a co-registration process to normalize sizes and coordinates in each image; and
performing a segmentation process to isolate a target region of the brain in each image.

4. The brain imaging system according to claim 3, wherein the co-registration process further includes:
obtaining a target brain atlas from a plurality of reference brain atlases built from one or more representations of brain;
spatially normalizing the brain of each image to a coordinate system; and
registering the brain of each image to the target brain atlas by matching anatomy of the brain with a representation of anatomy in the target brain atlas.

5. The brain imaging system according to claim 1, wherein the image enhancing process includes:
applying a contrast-limited adaptative histogram equalization (CLAHE) algorithm on each image to locally enhance differences between normal regions and regions of interest.

6. The brain imaging system according to claim 5, wherein the image enhancing process further includes:
performing a particle swarm optimization algorithm, before applying the CLAHE algorithm, to obtain optimal parameters for the CLAHE algorithm; and
applying the CLAHE algorithm on each image by utilizing the optimal parameters.

7. The brain imaging system according to claim 1, wherein the first deep learning model and the second deep learning model are a first long short-term memory (LSTM) neural network and a second LSTM neural network capable of learning order dependence in fitting time-series data.

8. The brain imaging system according to claim 1, wherein the processor is further configured to:
detecting a vessel occlusion, infarction or ischemia region of the first brain image set according to the plurality of brain perfusion indices.

9. The brain imaging system according to claim 8, wherein the plurality of brain perfusion indices include one or more of a first concentration curve, a first cerebral blood flow, a first cerebral blood volume, a first cerebral blood mean transit time and a first contrast agent time to peak.

10. The brain imaging system according to claim 1, wherein the processor is further configured to:
select, according to type of the one or more target brain lesions, the third deep learning model from a plurality of candidate deep learning models having been trained for identifying different types of brain lesions,
wherein the plurality of candidate deep learning models are trained by a plurality of training sets having different types of images, respectively.

11. The brain imaging system according to claim 10, wherein for each type of the brain lesions, the candidate deep learning models are trained by the different types of images, and the trained candidate deep learning models are each tested to determine whether or not each of the candidate deep learning models can be selected to identify the one or more target brain lesions.

12. The brain imaging system according to claim 11, wherein the one or more target brain lesions includes one or more of infarction areas, tumors, tumor metastasis, lymph nodes and lesions associated with dementia.

13. A brain imaging method, comprising:
configuring a first imaging device to capture a first brain image set by scanning a patient, wherein the first brain image set includes a plurality of first brain images that provides cerebral data representing a first contrast agent in a brain of the patient over time;
configuring a second imaging device to capture a second brain image set by scanning the patient, wherein the second brain image set includes a plurality of second brain images that provides cerebral data representing a second contrast agent in the brain of the patient over time; and
configuring a processor, which is electrically connected to the first imaging device and the second imaging device, to:
obtain, by performing an image pre-processing process on the first brain image set and the second brain image set, a first processed brain image set and a second processed brain image set;
obtain, by performing an image enhancing process on the first processed brain image set and the second processed brain image set, a first enhanced brain image set and a second enhanced brain image set;
select, by using a first deep learning model having been trained, first features from the first enhanced image set that are optimal for estimating cerebral perfusion;
select, by using a second deep learning model having been trained, second features from the second enhanced image set that are optimal for brain lesion identification;
obtain, by performing calculations on the first features, a plurality of brain perfusion indices; and
identify, by inputting the second features to a third deep learning model having been trained, position information and volume information of one or more target brain lesions in the brain of the patient.

14. The brain imaging method according to claim 13, wherein the first imaging device is a computed tomography (CT) imaging device, the plurality of first brain images are CT brain images, the second imaging device is a magnetic resonance imaging (MRI) device, and the plurality of second brain images are MRI brain images.

15. The brain imaging method according to claim 14, wherein the pre-processing process includes:
performing a re-alignment process to align positions of the brain in each image;
performing a co-registration process to normalize sizes and coordinates in each image; and
performing a segmentation process to isolate a target region of the brain in each image.

16. The brain imaging method according to claim 15, wherein the co-registration process further includes:
obtaining a target brain atlas from a plurality of reference brain atlases built from one or more representations of brain;
spatially normalizing the brain of each image to a coordinate system; and
registering the brain of each image to the target brain atlas by matching anatomy of the brain with a representation of anatomy in the target brain atlas.

17. The brain imaging method according to claim 13, wherein the image enhancing process includes:
applying a contrast-limited adaptative histogram equalization (CLAHE) algorithm on each image to locally enhance differences between normal regions and regions of interest.

18. The brain imaging method according to claim 17, wherein the image enhancing process further includes:
performing a particle swarm optimization algorithm, before applying the CLAHE algorithm, to obtain optimal parameters for the CLAHE algorithm; and
applying the CLAHE algorithm on each image by utilizing the optimal parameters.

19. The brain imaging method according to claim 13, wherein the first deep learning model and the second deep learning model are a first long short-term memory (LSTM) neural network and a second LSTM neural network capable of learning order dependence in fitting time-series data.

20. The brain imaging method according to claim 13, further comprising:
configuring the processor to detect a vessel occlusion, infarction or ischemia region of the first brain image set according to the plurality of brain perfusion indices.

21. The brain imaging method according to claim 20, wherein the plurality of brain perfusion indices include one or more of a first concentration curve, a first cerebral blood flow, a first cerebral blood volume, a first cerebral blood mean transit time and a first contrast agent time to peak.

22. The brain imaging system according to claim 13, further comprising configuring the processor to:
select, according to type of the one or more target brain lesions, the third deep learning model from a plurality of candidate deep learning models having been trained for identifying different types of brain lesions,
wherein the plurality of candidate deep learning models are trained by a plurality of training sets having different types of images, respectively.

23. The brain imaging system according to claim 22, wherein for each type of the brain lesions, the candidate deep learning models are trained by the different types of images, and the trained candidate deep learning models are each tested to determine whether or not each of the candidate deep learning models can be selected to identify the one or more target brain lesions.

24. The brain imaging system according to claim 23, wherein the one or more target brain lesions includes one or more of infarction areas, tumors, tumor metastasis, lymph nodes and lesions associated with dementia.
